(19) United States
(12) Patent Application Publication
(10) Pub. No.: US 2026/0248506 A1
(43) Pub. Date: Aug. 27, 2026

(54) WOUND ADHESIVE DEVICE AND METHODS OF USE THEREOF

(71) Applicants: Adam M. Rotunda, Newport Beach, CA (US); Carl F. Schanbacher, Boston, MA (US)

(72) Inventors: Adam M. Rotunda, Newport Beach, CA (US); Carl F. Schanbacher, Boston, MA (US)

(21) Appl. No.: 19/363,423

(22) Filed: Oct. 20, 2025

Related U.S. Application Data
(60) Provisional application No. 63/763,702, filed on Feb. 26, 2025.

(51) Int. Cl. A61B 17/08

(52) U.S. Cl. CPC A61B 17/085; A61B 2017/086

(57) ABSTRACT

Methods and membranes are disclosed for healing one’s body wounds, such as surgical wounds that may result from Mohs micrographic surgery. One or more membranes comprising membrane materials may be adhered to skin proximal to the body wound. The membrane(s) may be placed such that any reinforcing fibers of the membrane material(s) are generally parallel to a longitudinal axis of the body wound. The body wound may then be closed with sutures which may burrow into and out of the membrane material(s). There may be a connector which can increase and maintain tension between the membrane materials. After a period of time, such as at least 14 days, the sutures can be removed but the membrane(s) may remain on the skin. After another period of time has passed, the body wound may heal such that only a faint scar remains.

### CROSS-REFERENCE TO RELATED APPLICATIONS

This application relates to and claims the benefit of U.S. Provisional Application No. 63/763,702 filed February 26, 2025, and entitled “WOUND ADHESIVE DEVICE,” the entire disclosure of which is hereby wholly incorporated by reference.

### STATEMENT RE: FEDERALLY SPONSORED RESEARCH/DEVELOPMENT

Not Applicable

### BACKGROUND

### 1. Field of Invention

The present application relates to healing body wounds, such as surgical wounds that may result from procedures like Mohs micrographic surgery (MMS). More specifically, the present application relates to fibrous membranes which can be placed on regions of a user’s skin proximal to a body wound to be healed. The membranes may remain on the skin for a period of time while they keep the wound closed, leading to an improved scar healing process.

### 2. Related Art

It can prove challenging to repair Large Mohs micrographic surgery (MMS) wounds in high tension areas of a patient, like their distal leg. This problem is exacerbated when the patient is elderly, as their leg skin atrophies with age and becomes prone to suture tearing, or when the patient has photodamaged skin. While the repair methods traditionally suggested to these patients include healing by second intention, full thickness graft, or split thickness graft, most patients wish to avoid a possible graft failure. Other methods include a keystone flap or purse-string repair, but it would be ideal to minimize further incisions and instead have an option that provides a fast recovery that results in a predictable, linear scar. Currently, techniques meeting all or most of a patient’s desires are few and far between. As such, new and improved methods and membranes are desired in the art which allow for a relatively unobtrusive and efficient wound healing process that would result in faint scars.

### BRIEF SUMMARY

To solve these and other problems, methods and membranes are disclosed for obtaining improved body wound healing capabilities that may ultimately result in a faint, linear scar. One or more membranes, each comprising a membrane material and an adhesive, may be provided. The membrane material may be comprised of a nonwoven polyester material, such as Hypafix^{®}, while the adhesive may comprise a silicone-based medical adhesive, a hydrocolloid adhesive, a polyurethane-based adhesives, any other type of acrylic or acrylate adhesive (including polyacrylate and cyanoacrylates), or combinations thereof. A method for healing a body wound, such as a surgical wound that resulted from Mohs micrographic surgery (MMS), may comprise the steps of applying a skin cleaner and/or skin tackifier to a first and second region of the skin proximal to the body wound. Optionally, the body wound may be at least partially closed with a set of preliminary sutures. For instance, these preliminary sutures may close at least two thirds of the length of the body wound along the longitudinal axis; even more particularly, this could comprise a closure of at least one third of the length of the body wound along the longitudinal axis at a proximal end of the body wound and a similar closure at a distal end of the body wound.

Following these preparatory steps, a first membrane may be placed on the aforementioned first region and a second membrane may be placed on the aforesaid second region. The membranes may be placed such there is at least one membrane on either side of a longitudinal axis of the wound, which may be the axis upon which the wound is to be closed, and if the membrane material is a fibrous material having reinforcing fibers, the placement of the membranes may result in the reinforcing fibers being substantially parallel to the longitudinal axis of the body wound. Additionally, the membranes may be placed on their respective regions of the skin such that the shortest distance between an edge of the first membrane, the second membrane, or both, and an edge of the body wound is at most 10 mm.

Next, the body wound may be at least partially, more preferably completely, closed with a set of primary sutures. These primary sutures, in addition to burrowing into and out of the skin, may burrow into and out of the membranes, and these sutures may run generally perpendicular to the membrane materials’ longitudinally-oriented reinforcing fibers. At this stage, a first period of time may be allowed to pass, such as at least a day or at least 14 days, during which the wound can be kept largely dry. After this period of time has passed, the primary sutures may be removed, although the membranes may remain on the skin for a second period of time (e.g., at least a day), after which the membranes may be removed. It has been found that by keeping the membranes on the skin for this extra period of time, the body wound may heal considerably and result in a faint, inconspicuous scar.

Alternative embodiments are also envisioned for the membrane which may aid in the process of placing the membranes on the skin or healing the wound. Such membranes may further comprise a connector capable of engaging with a top surface of at least one membrane material. In some embodiments, this connector may be a rigid piece or sheet of plastic which connects two membrane materials in order to keep them apart at an adjustable or fixed distance (e.g., about 15 mm) so that they can be more easily placed upon the skin in a desired spatial relationship to the body wound. Afterwards, the connector can be removed from the membrane materials and the remainer of the method may be carried out substantially the same as described above.

Another type of membrane may use a connector in order to increase and maintain tension between two or more membrane materials. This may be a result of the connectors being capable of engaging with the top surfaces of the membrane materials, such as via a hook-and-loop fastener mechanism. In particular, the membrane materials may have top surfaces containing hooks, and the connectors may have corresponding loops such that after the membranes are placed on either side of a body wound, the connectors may be placed such that they engage with the top surfaces of the membrane materials and thus increase and maintain tension between those membrane materials. Such an embodiment may remove a need for, or alternatively act alongside, any sutures for closing the body wound.

In a final type of embodiment contemplated, a connector, having a surface with at least a first section containing loops and a second section containing hooks of a hook-and-loop fastener, is engageable with at least a first membrane material, while a second membrane material has a structural loop which defines a space operative to receive the connector. The connector could be stretched across a body wound, threaded through this space, and folded back over such that at least a portion of the first and second sections engage with one another. Such a mechanism may be particularly effective at increasing and maintaining tension between the membrane materials.

All of these embodiments are contemplated to be within the scope of this disclosure. These and other embodiments will become readily apparent to those skilled in the art from the following detailed description of the preferred embodiments having reference to the attached figures, the disclosure not being limited to any particular preferred embodiment.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a photograph of a patient’s body wound which arose after three stages of Mohs micrographic surgery (MMS);

FIG. 2 is a photograph of the same wound from FIG. 1, now being partially closed with sutures and having membranes of a first embodiment placed on the patient’s skin on either side of the wound;

FIG. 3 illustrates a top view of membranes of a second embodiment having a top surface with hooks corresponding to a hook and loop fastener which are being placed on regions of a user’s skin proximal to an open body wound;

FIG. 4 shows the top view of FIG. 3, in which connectors having a bottom surface with loops corresponding to a hook and loop fastener are being placed such that they engage with the top surfaces of the membrane materials to maintain tension between the membrane materials and partially close the body wound;

FIG. 5 depicts the top view of FIG. 4 in which the body wound has been closed by the placement of connectors;

FIG. 6 illustrates a side cross sectional slice view of FIG. 4;

FIG. 7 shows a side cross sectional slice view of FIG. 5;

FIG. 8 depicts a top view of a body wound having membranes of a third embodiment placed proximal to the wound and which have connectors having a top surface with a section having hooks and a section having loops of a hook and loop fastener, with each of the connectors being threaded through a space defined by structural loops;

FIG. 9 shows the top view of FIG. 8 in which the connectors have been folded such that the hooks and loops on the top surface of the connectors engage with each other in order to maintain tension between the membranes;

FIG. 10 is a photograph of the wound of FIG. 2, now having been closed throughout with sutures; and

FIG. 11 is a photograph of the wound from FIG. 10 after the sutures and membranes have been removed and 3 months after the MMS operations.

Common reference numerals are used throughout the drawings and the detailed description to indicate the same elements.

### DETAILED DESCRIPTION

Methods and membranes are disclosed for enhancing the closing and healing of one’s bodily wounds, including surgical wounds. A membrane may be comprised of a membrane material and an adhesive. After optionally cleaning the skin around one’s wound and/or at least partially closing the wound with sutures, one or more membranes may be placed on skin proximal to the wound. In particular, the membrane(s) may be placed such that membrane material is adhered to a region of the skin surrounding the wound. If the membrane material is a fibrous material having reinforcing fibers, the membrane(s) may be placed such that these reinforcing fibers are placed parallel to a longitudinal axis of the wound. The wound may then be closed with sutures. After a certain period of time, the sutures may be removed from the wound, but the membranes may remain on the skin. After another period of time has passed, the membranes may be removed. It is believed that the membranes maintain the structural integrity of the skin and prevent skin and integument tearing, as these methods have led to an enhanced skin healing process which has resulted in the wound turning into a faint scar, whereas alternative prior art methods may result in a more conspicuous scar.

This description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

The following methods and membranes may be used for healing a variety of body wounds. This includes wounds which may result from surgeries, emergencies, or traumatic experiences, or wounds which one may incidentally incur in everyday life. In particular, the methods and membranes contemplated herein have shown effective results when healing large Mohs micrographic surgery (MMS) wounds, lacerations, traumatic wounds, bites, tears, and contusions in high tension areas of a patient’s skin, such as the distal leg, scalp, trunk, and buttocks, even with patients having susceptible skin like elderly patients and/or patients with photodamaged skin. As people age, their leg skin atrophies and becomes prone to suture tearing when using conventional wound-closing methods, even when those sutures have minimal suture tension. By employing the methods and membranes of this present disclosure, such patients may reap the benefits of a fast, efficient wound healing process that can result in a faint, linear scar. Other categories of wounds which the present disclosure may help treat include traumatic lacerations and avulsions (e.g., from construction, industrial, kitchen, and agricultural work settings), miliary and combat related wounds (e.g., from gunshot wounds, shrapnel, bladed weapons, friction and impact, and animal/environmental trauma), those relate to emergency medical services and prehospital care (e.g., from motor vehicle accidents, worksite trauma, pedestrian accidents, hiking accidents, and thermal trauma/burn-associated lacerations), wounds resulting from other types of surgical procedures (e.g., trauma surgery wounds, orthopedic surgical incisions, laparoscopic port sites, and c-section and obstetric incisions), wounds treated through remote and austere medicine (as may be the case for expedition and wilderness medicine, underwater and marine wounds, aerospace and spaceflight trauma, and disaster medicine and humanitarian relief), pediatric and geriatric wounds (e.g., pediatric lacerations from playground activities and geriatric skin tears due to atrophic skin prone to break down), and high-tension and difficult-to-close wounds (e.g., scalp lacerations, joint and extremity wounds, and lower extremity lacerations). The membranes and methods disclosed herein may be adapted to be suitable for application in a particular context.

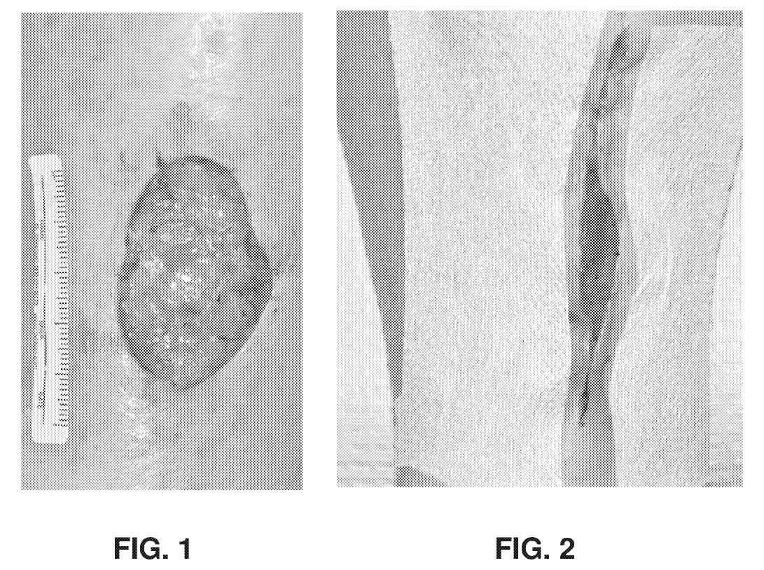

Referring now to FIG. 1, a patient’s body wound is shown. This body wound is approximately 3.0 by 4.2 cm and extends into the subcutaneous fat on the patient’s anterior left tibia. This wound arose after three stages of MMS. While the methods and membranes of this disclosure may be effective at healing these types of wounds, it will be appreciated that other types of wounds and wounds of differing geometries may be healed by the methods and membranes contemplated in this disclosure.

Any of the steps discussed in this disclosure could be performed by the person themselves who has the body wound to be healed, a third party like a doctor, surgeon, or other medical technician, or a combination thereof. It may be suitable for the person having the wound to heal themselves if their wound is minor or the result of an accident from everyday life; for example, someone who is hiking and accidentally cuts their leg on a rock may be capable of practicing the methods disclosed herein if they have proper membranes with them. Alternatively, if the wound is a result of a patient undergoing a surgical procedure, a doctor or other medical technician may perform most, if not all, the steps of a method related to the use of a membrane. A same patient may perform some of these steps at the direction of their doctor.

Prior to placing any membranes upon the skin, the skin around the wound may be cleaned with a skin cleaner like, for example, isopropyl alcohol and/or chlorhexidine, although other types of conventional and future developed skin cleaners may also be used. The skin may be allowed to air dry following said skin cleaning. Afterwards, or alternatively, a liquid skin tackifier can be applied to the dried skin to ensure greater adhesion of the membrane materials to the skin and reinforcement of the membrane materials itself. This liquid skin tackifier may be Mastisol^{®}, manufactured by Ferndale Laboratories in Ferndale, Michigan, which is comprised of ethanol, acetone, methyl salicylate, gum mastic, storax, and water; however, other conventional and any future developed liquid skin tackifiers may also or alternatively be used, such as Skin Tac^{TM} and Cavilon^{TM}, and acrylic-based skin tackifiers, like cyanoacrylates. The regions of skin which are cleaned and/or have skin tackifier applied to are ideally the same regions of skin in which the membrane material is to be applied upon.

Turning now to FIG. 2, the same wound from FIG. 1 is shown, now being partially closed with sutures and having membranes of a first embodiment placed on the patient’s skin on either side of the wound. Optionally, a body wound may be partially closed prior to placing one or more membranes on the skin proximal to the wound, and sutures may be utilized to close the wound in this manner. These particular sutures may be referred to as preliminary sutures. Those skilled in the art will be readily familiar with how a body wound may be optimally closed in order to achieve the most effective or desirable healing results, which may come in the form of the resulting scar on the patient’s skin being as small and inconspicuous as possible. In this respect, a surgeon operating on the wound or other type of user may decide how they wish to close the wound; this may, in turn, define a longitudinal axis of the body wound. As shown in FIG. 2, the manner in which the wound is being closed may result in this longitudinal axis being in an up-down direction in relation to the photograph shown. Depending on the nature of the wound and the way in which the wound is chosen to be closed, the longitudinal axis may not be a straight axis or an approximately straight axis, like the one shown in FIG. 2, but may be more curved or have sections in which the axis is straight and other sections in which the axis is curved.

Particular regions of a wound may optionally be closed with preliminary sutures prior to placing one or more membranes on the skin proximal to the wound. The extent to which a wound may be closed in this manner may be measured in relation to a longitudinal length of the wound upon the longitudinal axis. The length of an open wound along the longitudinal axis may be measured in order to approximate, for example, what regions of a wound correspond to a third of its length. It is contemplated that at least a third of the wound on either side of the wound along the longitudinal axis may be closed in this manner. For example, the wound depicted in FIG. 2 shows a wound whose proximal and distal thirds have been closed with deep (e.g., percutaneous), 3-0 polydioxanone sutures, while leaving the central third of the wound open. Other specified regions of a wound along the longitudinal axis may be specified and closed in this manner, and those skilled in the art would be familiar with how a wound would be partially closed at this stage in order to obtain the most desirable healing results.

Prior to placing the membrane(s) on the skin proximal to a wound, the membrane(s) may optionally be steam sterilized. Depending on the membrane material, steam sterilization may not compromise the tear-through strength of the membrane material, thus not substantially impeding its performance.

One or more membranes may be placed on the skin proximal to the wound. These membranes may be comprised of a membrane material and an adhesive on a bottom surface of the membrane material, which may be the same surface that engages with the skin. The membrane material can be a fibrous material, such as a nonwoven material, and may contain reinforcing fibers. In particular, membranes comprised nonwoven polyester materials, such as Hypafix^{®}, manufactured by BSN Medical Inc. in Charlotte, North Carolina, have been shown to yield remarkable scar healing results. That said, alternative membrane materials having similar capabilities may also be utilized in the methods and membranes discussed herein; this includes membranes known in the art and membranes which may be conceived of in the future. Other desirable properties a membrane material can have include stretchability, adaptation to body contours when placed on the skin, light compression when placed on the skin, and minimal constriction or congestion when placed on the skin. The adhesive may be a non-allergenic self-adhesive, like polyacrylate. The adhesive may further include, or alternatively be, a silicone-based medical adhesive, a hydrocolloid adhesive, a polyurethane-based adhesives, any other type of acrylic or acrylate adhesive (including cyanoacrylates), or combinations thereof. Acrylic-based adhesives may be preferred if the patient is expected to be exposed to a high-humidity environment while the membrane material remains on their skin, as acrylic-based adhesives typically perform better in such environments. Silicone adhesives may be preferred over acrylic adhesives if the patient has sensitive skin and/or is allergic to acrylics. Polyurethane-based adhesives may be highly water resistant and can provide good stretchability for flexible movement. The adhesive may be autoclaved (i.e., heat sterilized) prior to use of the membrane, and the adhesive itself may be water impervious, and/or have antimicrobial properties, which may be a result of the adhesive containing zinc or iodine. The adhesive need not be reinforced by another adhesive.

The membranes may be placed on skin very close to the wound, such that the shortest distance between an edge of a membrane and an edge of the wound is at most 10 mm, or at most 5 mm, for instance. The example shown in FIG. 2 shows two 2-inch wide Hypafix^{®} membranes which were applied approximately 3-5 mm from the edge of the wound. The example shown in FIG. 2 shows two membranes, each of which were placed on either side of the wound’s longitudinal axis, but there could be multiple membranes placed on either side of the longitudinal axis. The membrane may also extend a distance above and below a wound’s superior and inferior wound edges; in the example shown in FIG. 2, the Hypafix^{®} membranes extend about 1 cm above and below the wound’s superior and inferior edges.

If the membrane material has reinforcing fibers, as may be the case when using Hypafix^{®} as the membrane material, the membrane may be placed on the skin such that the reinforcing fibers are approximately parallel with the longitudinal axis of a wound. It has been found that this arrangement provides increased tear-through resistance in comparison to when these reinforcing fibers are placed more perpendicular to the longitudinal axis of a wound. The reinforcing fibers may be linearly spaced from one another within a membrane material, as is the case with Hypafix^{®}, such that the reinforcing fibers may be largely parallel to a wound’s mostly linear longitudinal axis. Alternatively, a membrane material may have reinforcing fibers that are curved or have sections in which the reinforcing fibers are linear and sections where they are curved, and these membranes may be specially designed such that their reinforcing fibers run parallel a longitudinal axis of an oddly shaped body wound that is not linear throughout.

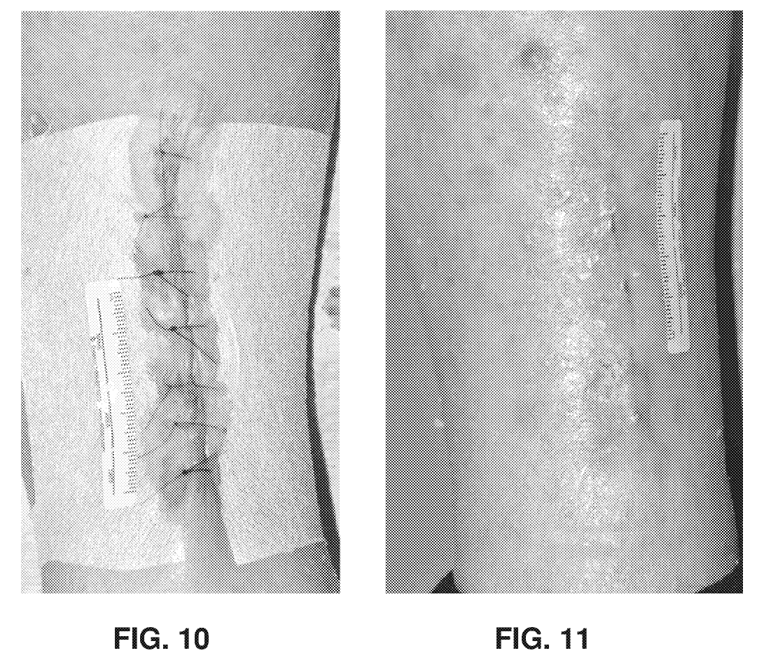

The membranes of this present disclosure may be of a first embodiment, as shown in FIG. 2, which do not contain additional features to increase or maintain tension between membranes on opposite sides of a body wound. Such membranes may nonetheless be capable of healing a body wound effectively, as will be shown in relation to the discussion of the photographs in FIGS. 10 and 11 towards the end of this disclosure. However, it is also contemplated that a membrane may be specially designed for use in the methods disclosed herein so as to facilitate a proper placement of the membrane material in relation to the wound and/or heal the wound more effectively. The following paragraphs will describe alternative embodiments of membranes which may fit such a purpose. It is worth noting that the discussions above regarding possible body wounds, the placement of membranes relative to a body wound, the optional use of preliminary sutures, etc. are equally applicable with these alternative membranes.

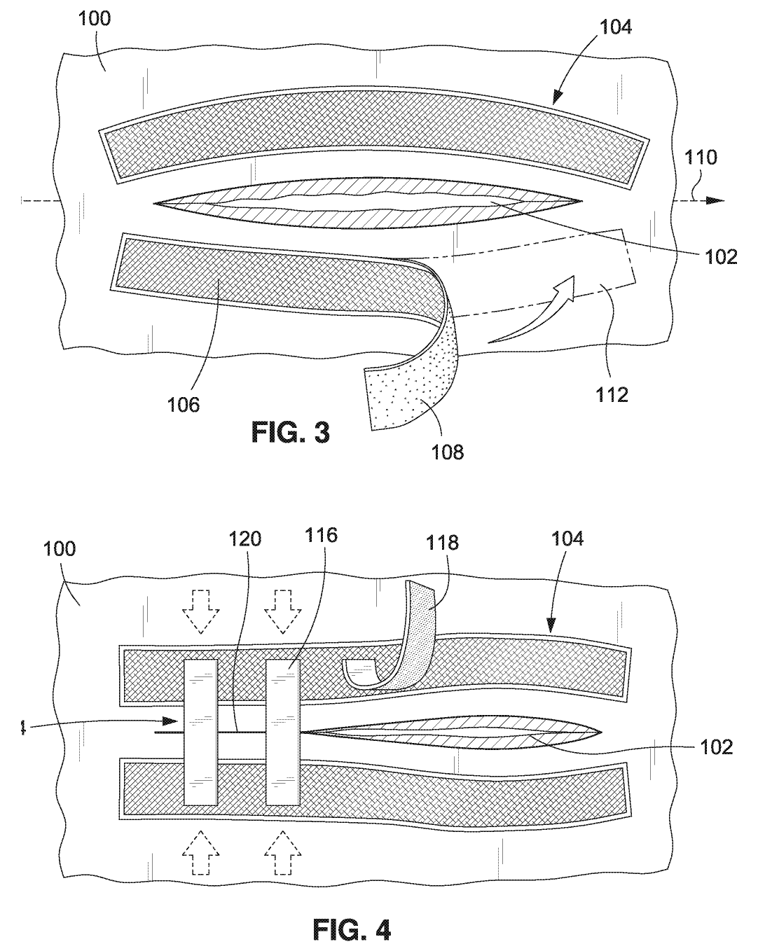

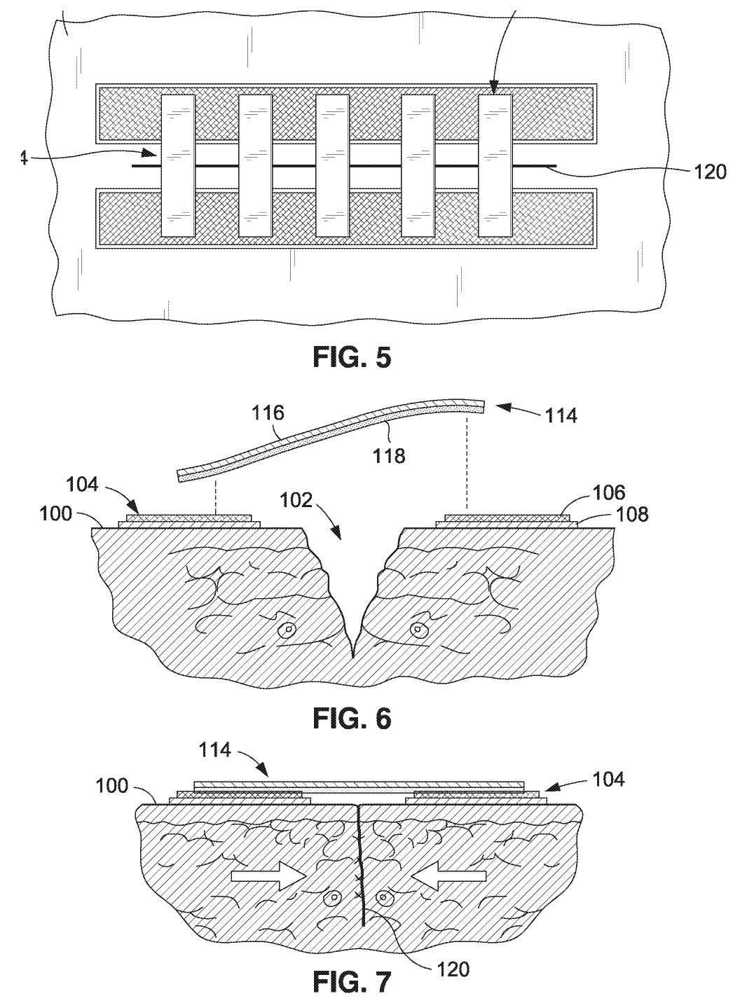

In one type of alternative embodiment, a membrane may be comprised of two or more membrane materials and individual adhesives associated with each membrane, wherein the membranes are connected to each other with a connector. This could come in the form of the connector being engaged with, for instance, the top surface of any membrane materials present, which could be the result of adhesion, snap-fit connection, threaded fasteners, etc., although preferably this will come in the form of a hook and loop fastener such as Velcro^{®}, as will be discussed later in relation to FIGS. 3-9.

The connectors may serve to ensure that the membranes are spaced from one another at a particular distance such when the membrane is placed on the skin, the membranes may be placed in a preferred close proximity to the edges of the wound (e.g., the connector may keep the membranes approximately 15 mm apart from each other). In this respect, the membrane may be designed such that the connector maintains an approximate distance between the membrane materials for a particular or known type of wound geometry, or the connector may be designed to allow user to adjust the distance between the membrane materials so that a membrane may be used for a variety of wound geometries. In such embodiments, the connector may be a rigid component.

A connector may be removable from the membranes. In such embodiments, the membrane may be placed on the skin such that the membranes materials are placed in a position relative to the wound, after which the connector may be removed. In this respect, the connector could be a plastic connector which may be simple to remove, and could take the form of, for example, one or more rigid, thin plastic bridges or a thin sheet of plastic. Removal of a connector after placement of a membrane in proximity to a wound may result in membrane materials being disconnected from each other and being emplaced on either side of a wound, leaving the wound in between the membrane materials easy to access, similar to the example illustrated in FIG. 2.

Alternatively, the connector may be intended to remain on the top surfaces of the membrane materials to aid in healing the wound. This type of connector may be configured so that a user may selectively increase tension between the membrane materials. This class of connector could come in the form of one or more fasteners (e.g., a zipper), wherein the connector may be fastened in order to increase and maintain tension between the membrane materials and thereafter loosened to decrease that tension, as desired. This category of connector may make it challenging to access the wound when fastened, as the connector may partially block the wound; if this is the case, the wound could be closed, as will be discussed in relation to FIG. 10, before the connector is placed in the tension configuration.

A connector could be capable of engaging with to a top surface of one of the membrane materials while in an unfastened state. Fastening such a connector may entail the connector engaging with a feature associated with a top surface of another membrane material which may be on the opposite side of a body wound. For example, in one embodiment it is contemplated that while loose, a connector may be engaged with the top surface of one membrane material via the top surface of the membrane material having hooks which interface with loops of a surface of a connector like a hook-and-loop fastener, such as Velcro^{®}. A second membrane material may also have a top surface having hooks of a hook-and-loop fastener, and the connector may also engage with this top surface in order to increase and maintain tension between the membrane materials. Although preferably the top surfaces of the membranes have hooks and the connector has corresponding loops, it is contemplated that the membranes may instead have the loops while the connector has the corresponding hooks, or one membrane’s surface has hooks while the other membrane’s surface has loops and the connector has a section with hooks and another section with loops.

One or more connectors may initially be separate components from any membranes and come packaged therewith. This may allow one to place the membranes on the skin and thereafter place the connector to maintain tension between the membrane materials.

Turning now to FIG. 3, membranes 104 of a second embodiment having a top surface 106 with hooks corresponding to a hook and loop fastener which are being placed on regions 112 of a user’s skin 100 proximal to an open body wound 102 is illustrated. The membranes 104 illustrated may, like the membranes discussed above in relation to FIG. 2, comprise a membrane material and an adhesive on a bottom surface 108 of the membrane material. The membranes 104 shown here in FIG. 3 further have features which may facilitate interaction with separate connector pieces. In this embodiment, the top surface 106 of the membrane materials have hooks corresponding to a hook-and-loop fastener, such as those associated with Velcro^{®}. As shown, the manner in which the body wound is being closed may define a longitudinal axis 110.

Referring now to FIGS. 4, 5, 6, and 7, the membranes of FIG. 3, now having connectors 114 placed to fasten the membranes 104 and facilitate closing the body wound, are shown, with FIGS. 6 and 7 showing side cross sectional slice views of the body wound. The connectors 114 may have a top surface 116 and a bottom surface 118 that is designed to interact with the features of the top surface 106 of the membrane materials. In these figures, this comes in the form of the bottom surface 118 of the connectors 114 having loops which may fasten with the hooks of the top surface 106 of the membrane materials. Multiple connectors 114 may be provided to maintain tension between the membrane materials, as is shown in FIGS. 4-7, and regions of an open body wound 102 may be pushed together prior to placing these connectors 114 such that the wound starts to become a closed body wound 120. This connector arrangement may ease or improve the process of closing the body wound, which will be discussed in more detail in relation to the following FIG. 10, and/or result in improved scar healing capabilities. The connectors 114 may be sufficiently long in order to engage with two particular membranes 104 on either side of a body wound.

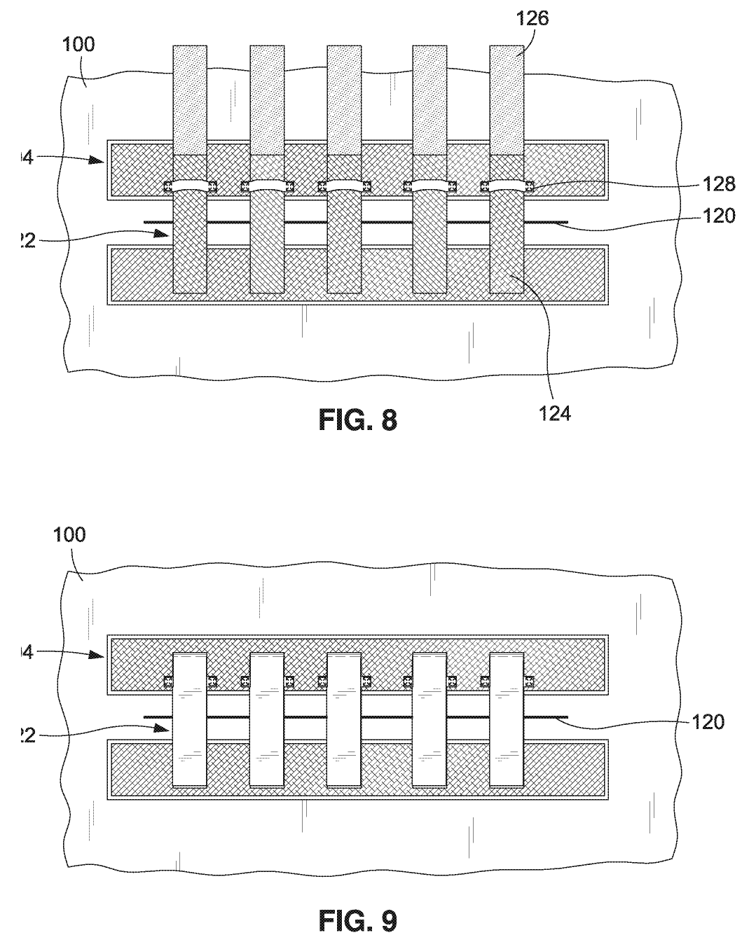

Referring now to FIGS. 8 and 9, a top view of a body wound having membranes of a third embodiment placed proximal to the wound is shown. The membranes 104 here may be substantially the same any of the membranes previously described, except that there may be one or more structural loops 128 associated with one or more of the membranes 104. These structural loops 128 may be embedded or otherwise engaged with a particular membrane 104 such that the structural loop 128 defines a space capable of receiving a unique connector 122. This connector 122 may be capable of engaging with one of the membranes 104, which could be a result of one of the engagement mechanisms previously discussed, and the connector may be engaged with a membrane 104 not having any structural loops 128. This connector 122 could further have a top surface comprised of at least a first section 124 and a second section 126. The first section 124 may have hooks corresponding to a hook and loop fastener, while the second section 126 may have loops corresponding to a hook and loops fastener, or vice versa. The connector 122 may be long enough such that lengths of the first section 124 and second section 126 may be sufficiently long to reach two membranes 104 positioned on either side of a body wound. The connector 122 can be threaded through the space defined by a structural loop 128, as is shown in FIG. 8. The connector 122 may be sufficiently flexible to allow the connector 122 to be folded such that at least a portion of the second section 126 may engage with the first section 124, as shown in FIG. 9, so that tension can be increased and maintained between the membranes 104. In this respect, this embodiment may function similar to shoes which use hook and loop fasteners to tighten the shoe, and it is contemplated that this embodiment may be even more effective healing a body wound due to further increased tension between the membranes.

Referring now to FIG. 10, the photograph of the body wound of FIG. 2 is shown, having now been closed throughout. The remaining disclosure, involving the closing and healing of the body wound, may be applicable to any of the membranes disclosed, including the embodiments discussed in relation to FIG. 3-9 above. After placing the membrane(s) on the skin, the wound may be closed with, for example, sutures. These sutures, which can be referred to as primary sutures, may be a different type of sutures to those which may have been used to partially close the wound before placing the membrane(s) on the skin (as discussed above in relation to FIG. 2). Unlike those preliminary sutures, the primary sutures may be placed through the longitudinal length of a body wound, or perhaps just the portions of the body wound which don’t already have sutures. Preferably, these primary sutures will also be placed such that the sutures run generally perpendicular to the membrane’s longitudinally-oriented reinforcing fibers. These primary sutures may also be emplaced in the skin such that they burrow into and out of the membranes. This open, high-tension structure may readily close the wound. The example shown in FIG. 10 used 4-0 CP-2 polypropylene epidermal sutures which span the superior end of the wound and proceeded inferiorly every 1 cm across the wound while burrowing into and exiting out of the Hypafix^{®} such that sutures is anchored perpendicularly to the reinforcing fibers of the Hyapfix^{®}.

In the event that the embodiment employed uses connectors which are meant to stay on the membranes and maintain tension while the wound heals, as is the case for the connectors 114, 122 discussed above in relation to FIGS. 3-9, the body wound does not need to be closed with these primary sutures, but alternatively the wound may be closed in this manner prior to the connectors becoming fastened (so that the sutures can be emplaced in the skin without the connectors potentially getting in the way).

Once the membranes and/or sutures are emplaced, a dressing may be applied upon the wound, such as a thin layer of petrolatum, although other suitable dressings may be used in addition to or alternatively to this dressing. Again, any connectors can be positioned on the membranes after this dressing is applied so that the presence of the connectors does not interfere with the application of the dressing. A period of time may be allowed to lapse, such as at least a day or at least 14 days, and preferably the wound may be kept largely dry during this period.

After this period of time has lapsed, any sutures may be removed from the wound; any connectors may be removed from their membranes in order to remove these sutures. However, any such connectors could again be positioned on their membranes, and the membrane(s) may remain on the skin for longer, at least a day, for instance. The wound may show little to no evidence of tearing or wound dehiscence after removal of these sutures, as was the case in the example displayed in the figures.

At any point while the membranes are on the skin, an overlay patch may be placed on a membrane to reinforce its attachment to the skin. Such overlay patches may include, but are not limited to, products like GrifGrips, Simpatch, and RockTape.

Turning now to FIG. 11, the wound from FIG. 10 is shown after the sutures and membranes have been removed. This photograph was taken three months after the MMS operations. The wound may have healed greatly and preserved lower extremity contour and mobility at this stage, as was the case for the wound shown in this FIG. 11. It is worth noting that the scar remaining on the patient’s skin is quite faint. Therefore, it can be appreciated that the membranes and methods of this present disclosure can provide improved and unobtrusive wound healing capabilities for a variety of body wounds which may result in small and inconspicuous scars on one’s skin.

The above description is given by way of example, and not limitation. Given the above disclosure, one skilled in the art could devise variations that are within the scope and spirit of this disclosure. Further, the various features of the embodiments disclosed herein can be used alone, or in varying combinations with each other and are not intended to be limited to the specific combination described herein. Thus, the scope of the claims is not to be limited by the illustrated embodiments. Additional modifications and improvements of the present disclosure may also be apparent to those of ordinary skill in the art. Thus, the particular combination of parts and steps described and illustrated herein is intended to represent only certain embodiments of the present subject matter and is not intended to serve as limitations of alternative devices and methods within the spirit and scope of this disclosure.

What is claimed is:

1. A method for healing a body wound, the method comprising the steps of:
placing a first membrane comprising a first membrane material and a first adhesive on a first region of skin proximal to the body wound;
placing a second membrane comprising a second membrane material and a second adhesive on a second region of skin proximal to the body wound;
at least partially closing the body wound with primary sutures;
allowing a first period of time to pass;
removing the primary sutures from the body wound after the first period of time has passed;
allowing a second period of time to pass;
removing the first membrane from the first region of skin after the second period of time has passed; and
removing the second membrane from the second region of skin after the second period of time has passed;
wherein said step of placing of the first membrane results in at least a portion of the first membrane to be placed on one side of a longitudinal axis of the body wound; and
wherein said step of placing of the second membrane results in at least a portion of the second membrane to be placed the other side of a longitudinal axis of the body wound.

2. The method of claim 1, wherein the body wound is a surgical wound.

3. The method of claim 2, wherein the body wound is associated with a Mohs micrographic surgical procedure.

4. The method of claim 1, wherein prior to said steps of placing the first and second membranes, the method further comprises a step of:
cleaning the first region of skin, the second region of skin, or both with a skin cleaner.

5. The method of claim 1, wherein prior to said steps of placing the first and second membranes, the method further comprises a step of:
applying a skin tackifier to the first region of skin, the second region of skin, or both.

6. The method of claim 1, wherein prior to said steps of placing the first and second membranes, the method further comprises a step of:
at least partially closing the body wound with preliminary sutures.

7. The method of claim 6, wherein the preliminary sutures close at least two thirds of a length of the body wound along the longitudinal axis of the body wound.

8. The method of claim 7, wherein the preliminary sutures close at least a third of the length of the body wound along the longitudinal axis at a proximal end of the body wound and at least a third of the length of the body wound along the longitudinal axis at a distal end of the body wound.

9. The method of claim 1, wherein the shortest distance between an edge of the first membrane, the second membrane, or both, and an edge of the body wound is at most 10 mm.

10. The method of claim 1, wherein the first membrane material, the second membrane material, or both are a nonwoven polyester material.

11. The method of claim 1, wherein the first membrane material, the second membrane material, or both are a fibrous material having reinforcing fibers; and wherein said steps of placing the first and second membrane results in the reinforcing fibers being substantially parallel to the longitudinal axis of the body wound.

12. The method of claim 1, wherein the first adhesive, the second adhesive, or both are polyacrylate.

13. The method of claim 1, wherein said step of closing results in at least some of the primary sutures to burrow into and out of the first membrane material, the second membrane material, or both.

14. The method of claim 1, wherein the first period of time is at least a day.

15. The method of claim 1, wherein the second period of time is at least a day.

16. A membrane for healing a body wound, the membrane comprising:
a first fibrous membrane material having reinforcing fibers, the first membrane material having a top surface and a bottom surface;
a first adhesive on the bottom surface of the first membrane material;
a second fibrous membrane material having reinforcing fibers, the second membrane material having a top surface and a bottom surface;
a second adhesive on the bottom surface of the second membrane material; and
a connector connected to the first membrane material and the second membrane material so as to define a distance between the first and second membrane materials.

17. The membrane of claim 16, wherein connector is configured to allow the distance to be adjusted.

18. The membrane of claim 16, wherein the connector is removable from the first and second membrane materials.

19. A membrane for healing a body wound, the membrane comprising:
a first fibrous membrane material having reinforcing fibers, the first fibrous membrane material having a top surface and a bottom surface;
a first adhesive on the bottom surface of the first fibrous membrane material;
a second fibrous membrane material having reinforcing fibers, the second fibrous membrane material having a top surface and a bottom surface;
a second adhesive on the bottom surface of the second fibrous membrane material; and
a connector operative to engage with the top surface of the first fibrous membrane material, the top surface of the second fibrous membrane material, or both.

20. The membrane of claim 19, wherein at least a section of a surface of the connector contains either hooks corresponding to a hook and loop fastener or loops corresponding to the hook and loop fastener.
